(19) United States
(12) Patent Application Publication
(10) Pub. No.: US 2002/0160502 A1
(43) Pub. Date: Oct. 31, 2002

(54) Recombinant BCG vaccines for the prevention and treatment of cancer

(75) Inventors: Maureen Angela Chung, Providence, RI (US); Surendra Sharma, Warwick, RI (US); Michael Alan O'Donnell, Iowa City, IA (US); Helena R. Chang, Los Angeles, CA (US)

Correspondence Address: LAHIVE & COCKFIELD, 28 STATE STREET, BOSTON, MA, 02109, US

(73) Assignee: Roger Williams Hospital, Providence, RI

(21) Appl. No.: 09/965,131

(22) Filed: Sep. 26, 2001

Related U.S. Application Data
(60) Provisional application No. 60/235,455, filed on Sep. 26, 2000.

(51) Int. Cl. A61K 39/02; C12N 1/21

(52) U.S. Cl. 435/252.3; 424/200.1; 424/264.1

(57) ABSTRACT

The present invention relates to recombinant mycobacteria, particularly recombinant M. bovis, BCG, engineered to express a MUC1 polypeptide and human interleukin-2 for use in cancer immunotherapy.

### RELATED APPLICATIONS

This application claims priority to U.S. Provisional Application No. 60/235,455 entitled “Recombinant BCG Vaccines Secreting mucin and interleukin-2” filed on Sep. 26, 2000, the contents of which are incorporated herein by reference.

### BACKGROUND OF THE INVENTION

Breast cancer is the most common malignancy in women (Landis, Sh et al. (1998) CA Cancer J. Clin. 48:6-29). Although the majority of women with breast cancer are diagnosed with local and/or regional disease only, approximately one quarter of these women will die of systemic disease. Their death from breast cancer occurs despite the use of combination systemic therapy consisting of hormonal agents and/or chemotherapy, suggesting the existence of microscopic systemic disease that is refractory to current modes of treatment.

Immunotherapy is a new area being investigated for the treatment or prevention of breast cancer. An efficacious vaccine may offer women with breast cancer a new form of adjuvant treatment, potentially without some of the side effects now encountered with current modes of systemic treatment. In addition, an efficacious cancer vaccine could be beneficial in two ways. First, it could be used to prevent the development of subsequent breast cancer by inducing ‘breast cancer immunity’ (i.e. women who survive their first encounter with breast cancer, and, who are at a significant risk for the development of a metachronous breast cancer (Heron, D. E. et al. (2000) Cancer 88:2739-2750)). Second, it could be used to “immunize” women who are at a significant risk for the development of breast cancer because of their family history or intrinsic genetic factors. Currently, the only option that these women have to decrease their risk for the development of breast cancer is bilateral prophylactic mastectomies or long-term hormonal treatment. Although many attempts have been made to develop such vaccines, none has been shown to be effective against breast cancer.

Immunobiologists have learned that a poor antigen (in terms of eliciting an immune response) can be turned into a strong antigen by changing the molecular environment. Changes of hapten carrier allow T cell helper cells to become active, making the overall immune response stronger. Thus, changing the carrier can also turn a tolerogenic antigen into an effective antigen. McBridge et al. (1986) Br. J. Cancer 53:707. Often the immunological status of a cancer patient is suppressed such that the patient is only able to respond to certain T-dependent antigens and not to other antigen forms. From these considerations, it would make sense to introduce molecular changes into the tumor associated antigens before using them as vaccines. Unfortunately, this is impossible to accomplish for most tumor antigens, because they are not well defined and are very hard to purify.

One of the candidate tumor associated antigens for breast cancer is MUCIN-1 (MUC1) protein. MUC1 protein is known by a variety of names including DF3 and episialin (Ligtenberg, M J et al. (1990) J. Biol. Chem. 265:5573-5578). The human MUC1 gene encodes for a large transmembrane polypeptide consisting of a variable number of tandem repeats (VNTR) of a twenty amino acid sequence (Siddiqui, J. et al. (1987) Proc. Natl. Acad. Sci. USA 85:2320-2323). MUC1 is expressed on a variety of epithelial derived cells including breast ductal cells. In the benign state, MUC1 is heavily glycosylated and its distribution is limited to the apical surface of the ductal cell (Croce, M V et al. (1997) Anticancer Res. 17:4287-4292). In the malignant state, there is increased expression of under-glycosylated MUC1 which is distributed along the entire cell surface (Irimura, T. et al. (1999) J. Biochem (Tokyo) 126:975-985). Under-glycosylation of MUC1 in breast malignancies unmasks novel epitopes on the protein that are unique to the malignant state, and make the protein a candidate tumor-associated tumor antigen (Burchell, J. et al. (1993) Epithelial Cell Biol. 4:155-162). MUC1, in its malignant form, has been shown to be immunogenic with each tandem repeat containing an epitope (Girling, A. et al. (1989) Int. J. Cancer 43:1072-1076). MUC1 antibodies have been detected in breast cancer patients, albeit at a low rate (Kotera, Y. et al. (1994) Cancer Res. 54:2856-2860). It has been postulated that a MUC1 vaccine may be capable of stimulating an immune response against tumor cells bearing this tumor associated antigen and in this manner be useful for breast cancer immunotherapy (Taylor-Papadimitriou, J. et al. (1999) Biochem Biophys Acta. 1455:301-313).

There have been several attempts to use MUC1 as a cancer vaccine. Most of the work has focused on the use of a synthetic peptide containing five of the tandem repeats, either by itself or conjugated to a carrier protein (Goydos, J S et al. (1996) J. Surg. Res. 63:298-304; Goilewski, T et al. (2000) Clin Cancer Res 6:1693-1701). These MUC1 vaccines have been able to stimulate a humoral MUC1 response.

However, despite the encouraging results obtained with these vaccines it would be preferable to use a live MUC1 construct as a vaccine. Attempts at using viral vectors have met with limited success (Bu, D et al. (1993) J. Immunother 14:127-135). The MUC1 protein expressed by host cells infected with MUC1-viral vectors is predominantly glycosylated mimicking the benign form of MUC1 protein. Additionally, the MUC1 proteins expressed are heterogeneous, suggesting a varying pattern of glycosylation or some instability in expression of the recombinant protein (Henderson, RA et al. (1998) J. Immunother 21:247-256).

Breast cancer is not curable by standard therapies. Even if a patient responds to traditional therapy, there is often a significant risk of recurrence. Thus, new therapeutic approaches for this disease are needed. The present invention overcomes the deficiencies in the prior art by providing recombinant bacterium engineered to secrete a Th1 cytokine and to express a tumor antigen that elicits an immune response in a subject.

### SUMMARY OF THE INVENTION

The invention relates, at least in part, to the treatment and/or prevention of cancer using a recombinant bacterium engineered to secrete a biologically active cytokine and to express a tumor antigen.

One aspect of the invention features a recombinant bacterium engineered to secrete a cytokine and to express a tumor antigen.

In one embodiment, the recombinant bacterium is an enterobacteria. In a preferred embodiment, the bacterium is E. coli.

In another embodiment, the bacterium is a mycobacterium. In a preferred embodiment, the mycobacterium is attenuated Mycobacterium bovis bacillus Calmette-Guerin (BCG) or Mycobacterium smegmatis.

In yet another embodiment of the invention, the recombinant bacterium secretes a Th1 cytokine. In a preferred embodiment, the cytokine is selected from the group consisting of GM-CSF, IFN-γ, TNF-α, IL-2, IL-12, IL-15 and IL-18. In a preferred embodiment, the secreted cytokine is IL-2.

In another embodiment of the invention, the recombinant bacterium is engineered to express a tumor antigen. In a preferred embodiment, the tumor antigen is selected from the group consisting of MUC1, CEA, oncofetal antigens and tumor-associated antigens. In a preferred embodiment, the tumor antigen is MUC1.

In another aspect, the invention features a recombinant mycobacterium comprising a first nucleic acid molecule encoding a Th1 cytokine operatively linked to a first promoter and a mycobacterial secretion signal sequence and a second DNA molecule encoding a tumor antigen operatively linked to a second promoter, wherein the cytokine is expressed and secreted from the recombinant mycobacterium and the tumor antigen is expressed by the mycobacterium, such that the mycobacterium is capable of inducing an immune response to the tumor antigen in a subject.

In one embodiment, the recombinant bacterium comprises a first promoter which is a bacterial heat shock protein (hsp) gene promoter or a bacterial stress protein gene promoter. In a preferred embodiment, the first promoter is hsp60or hsp70.

In another embodiment, the recombinant bacterium comprises a second promoter that is a bacterial heat shock gene promoter or a bacterial stress protein gene promoter. In a preferred embodiment, the first promoter is hsp60or hsp70.

In another embodiment, the recombinant bacterium comprises a bacterial secretion signal sequence. In a preferred embodiment, the bacterial secretion sequence is the BCG alpha antigen signal sequence.

In another embodiment of the invention, the subject is a mammal. In a preferred embodiment, the subject is human.

Another aspect of the invention features a recombinant mycobacterium which comprises (a) a first DNA molecule encoding a Th1 cytokine, (b) a first promoter, (c) a mycobacterial secretion sequence, (d) a second DNA molecule encoding a tumor antigen and (e) a second promoter wherein the 5′ to 3′ order is the first promoter of (b), the secretion signal sequence of (c), the first DNA molecule (a), the second promoter of (e), the second DNA molecule of (d), wherein the expression of the first DNA molecule of (a) is under the control of the first promoter of (b) and the cytokine is expressed and secreted from the mycobacterium and the expression of the second DNA molecule of (d) is under the control of the second promoter of (e) and the tumor antigen is expressed by the mycobacterium such that the recombinant mycobacterium is capable of inducing an immune response to the tumor antigen in a subject.

Another aspect of the invention features a recombinant BCG having a dual promoter plasmid comprising (a) a first DNA molecule encoding interleukin-2, (b) a first promoter, (c) a mycobacterial secretion signal sequence, (d) a second DNA molecule encoding MUC1 and (e) a second promoter wherein the 5′ to 3′ order is the first promoter of (b), the secretion signal sequence of (c), the first DNA molecule of (a), the second promoter of (e) and the second DNA molecule of (d), wherein the expression of the first DNA molecule of (a) is under the control of the first promoter of (b) and the cytokine is expressed and secreted from the mycobacterium and the expression of the second DNA molecule of (d) is under the control of the second promter of (e) and the tumor antigen is expressed by the mycobacterium thereby inducing an immune response to the tumor antigen in a mammalian host.

In another aspect, the invention features a recombinant bacterium encoding a tumor antigen and having enhanced immunostimulatory properties comprising a first DNA molecule encoding a cytokine and a second DNA molecule encoding a tumor antigen, wherein the first DNA molecule encoding the cytokine is under the control of a first promoter and the cytokine is secreted from the bacterium in a biologically active form and wherein the second DNA molecule encoding the tumor antigen is under the control of a second promoter and the tumor antigen is expressed by the recombinant bacteria.

In another aspect, the invention features an attenuated recombinant BCG encoding MUC1 and having enhanced immunostimulatory properties and having incorporated therein a plasmid comprising a first DNA molecule encoding interleukin-2 operably linked to a first mycobacterial heat shock protein gene promoter and a mycobacterial secretion signal sequence and a second DNA molecule encoding MUC1 operably linked to a second mycobacterial heat shock protein gene promoter wherein the 5′ to 3′ order of the plasmid is the first promoter, the secretion signal sequence, the first DNA molecule encoding the interleukin-2, the second promoter, the second DNA molecule encoding MUC1 wherein the interleukin-2 is expressed and secreted from the recombinant BCG in a biologically active form and the MUC1 is expressed by the recombinant BCG.

In another aspect, the invention features an E. coli-BCG shuttle plasmid which, when expressed in a mycobacterium, results in specificity for a tumor antigen and enhanced immunstimulatory properties, the shuttle plasmid comprises (a) a first DNA molecule encoding a Th1 cytokine, (b) a first promoter comprising DNA encoding a mycobacterial heat shock protein promoter and translational start site, (c) a mycobacterial secretion signal sequence, (d) a second DNA molecule encoding a tumor antigen, and (e) a second promoter comprising DNA encoding mycobacterial heat shock protein promoter and translational start site wherein the 5′ to 3′ order is the first promoter of (b), the secretion signal sequence of (c), the first DNA molecule of (a), the second promoter of (e) and the second DNA molecule of (d), wherein the expression of the first DNA molecule of (a) is under the control of the first promoter of (b) and the cytokine is expressed and secreted from the mycobacterium in a biologically active from and the expression of the second DNA molecule of (d) is under the control of the second promoter of (e) and the tumor antigen is expressed by the mycobacterium.

In one embodiment, the E. coli-BCG shuttle plasmid further comprises an epitope tag 5′ of the DNA encoding a TH1 cytokine. In a preferred embodiment, the epitope tag is viral influenza hemagglutinin.

In another embodiment, the E. coli-BCG shuttle plasmid further comprises an epitope tag 5′ of the DNA encoding a tumor antigen. In a preferred embodiment, the epitope tag is viral influenza hemagglutinin.

In another aspect, the invention features a method of inhibiting growth or proliferation of, or inducing killing of a tumor cell in a subject, comprising administering to the subject a recombinant bacterium of the present invention, including all the above-described aspects and embodiments, in an amount that is effective to inhibit growth or proliferation of, reduce the size of, or induce killing of the tumor in the subject.

In one embodiment, the subject is a mammal. In a preferred embodiment, the subject is a human.

In another aspect, the invention features a method of preventing the formation of a tumor in a subject comprising administering to the subject a recombinant bacterium of any of the present invention, including all the above-described aspects and embodiments, in an amount that is effective to prevent the formation of the tumor in the subject.

In yet another aspect, the invention features a method for stimulating an immune response to an immunogenic protein or fragment thereof, in a subject, comprising administering an effective amount of a recombinant bacterium of the present invention, including all the above-described aspects and embodiments, such that an immune response is stimulated against the immunogenic protein in the subject.

In yet another aspect, the invention features a method of treating a subject with cancer comprising administering to the subject an effective amount of the recombinant bacterium of the present invention, including all the above-described aspects and embodiments, such that the cancer is treated.

In one embodiment, the cancer to be treated is selected from the group consisting of breast cancer, prostate cancer, colon cancer, lung cancer, pancreatic cancer and ovarian cancer. In a preferred embodiment, the cancer to be treated is breast cancer.

In another aspect, the invention features a vaccine for immunizing a subject against a neoplastic disease, comprising a recombinant bacterium of the present invention, including all the above-described aspects and embodiments, and a pharmaceutically acceptable carrier therefor, wherein the recombinant bacterium in an amount effective to immunize a subject against a neoplastic disease.

In another embodiment. the invention features a pharmaceutical composition comprising a recombinant bacterium of the present invention, including all the above-described aspects and embodiments, and a pharmaceutically acceptable carrier.

In yet another embodiment, the invention features a kit for immunizing a subject against a neoplastic disease comprising the vaccine of the present invention, including all the above described aspects and embodiments, and instructions for use.

In yet another embodiment, the invention features a kit for treating cancer in a subject comprising the pharmaceutical composition of the present invention, including all the above-described aspects and embodiments, and instructions for use.

### BRIEF DESCRIPTION OF THE DRAWINGS

FIG. 1 shows a schematic representation of shuttle plasmid pMOD12.

FIG. 2 shows a schematic diagram of pIL2MUC1.

FIG. 3 shows a survival curve for tumor engrafted mice immunized with PBS, BCG-261, MUC1 or BCG-IL2MUC1.

FIG. 4 depicts the rate of tumor growth in mice immunized with PBS, BCG-261, MUC1 or BCG-hIL2MUC1.

FIG. 5 shows flow cytometery with anti-CD45 antibody of peritoneal lymphocytes obtained from mice vaccinated with PBS, MUC1 peptide, BCG-261 and BCG-hIL2MUC1.

FIG. 6 shows flow cytometry with anti-CD25 antibody demonstrating the relative expression of IL2 alpha receptor in splenic lymphocytes after vaccination with BCG-261 or BCG-hIL2MUC1.

### DETAILED DESCRIPTION OF THE INVENTION

I. Definitions

In describing the present invention, the following terms will be employed, and are intended to be defined as indicated below.

Mycobacterium bovis-BCG is an avirulent M bovis derivative which is widely used throughout the world and is commonly used to provide protection against tuberculosis, although its effectiveness has recently been called into question. Mycobacterium smegmatis is a nonpathogenic bacillus which shares antigenic and adjuvant properties with BCG. Both are reasonably easy to grow into culture.

Also used herein, the term “attenuated” relates to the dilution, lessening or elimination of virulence of an organism. The organism may include, but is not limited to, bacteria, viruses, fungi, parasites, and the like.

The term “tumor antigen” as used herein relates to any antigen expressed on a tumor cell, including but not limited to, Mucin1, chorioembryonic antigen (CEA), oncofetal antigens and tumor-associated antigens. Also included in this definition are any antigens expressed by tumor cells that are encoded by a single DNA strand.

The term “MUC1” or “MUCIN1” or “DF3” or “episialin” are used interchangeably and refer to the tumor antigen that encodes a large transmembrane polypeptide consisting of a variable number of tandem repeats (VNTR) of a twenty amino acid sequence. MUC1 is expressed on a variety of epithelial-derived cells including breast ductal cells. In the benign state, MUC1 is heavily glycosylated and its distribution is limited to the apical surface of the ductal cell. In the malignant state, there is increased expression of under-glycosylated MUC1 which is distributed along the entire cell surface. Under glycosylation of MUC1 in breast malignancies unmasks novel epitope on the protein that is unique to the malignant state and makes it a candidate tumor-associated tumor antigen.

The term “protein” is used herein to designate a naturally occurring polypeptide. The term “polypeptide” is used in its broadest sense, i.e., any polymer of amino acids (dipeptide or greater) linked through peptide bonds. Thus, the term “polypeptide” includes proteins, oligopeptides, protein fragments, analogs, muteins, fusion proteins and the like.

A DNA “coding sequence” or a “nucleotide sequence encoding” a particular protein, is a DNA sequence which is transcribed and translated into a polypeptide in a host cell when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A transcription termination sequence will usually be located 3′ to the coding sequence.

A coding sequence is “operably linked to” another coding sequence when RNA polymerase will transcribe the two coding sequences into mRNA, which is then translated into a chimeric polypeptide encoded by the two coding sequences. The coding sequences need not be contiguous to one another so long as the transcribed sequence is ultimately processed to produce the desired chimeric protein.

A “promoter sequence” is a DNA regulatory region capable of binding RNA polymerase in a cell and initiating transcription of a downstream (3′ direction) coding sequence. For purposes of defining the present invention, the promoter sequence is bound at the 3′ terminus by the translation start codon (ATG) of a coding sequence and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eucaryotic promoters will often, but not always, contain “TATA” boxes and “CAT” boxes. Procaryotic promoters contain Shine-Dalgarno sequences in addition to the −10 and −35 consensus sequences.

The term “treatment” as used herein refers to either (i) the prevention of tumor growth or regrowth of the tumor (prophylaxis), or (ii) the reduction or elimination of symptoms or the disease of interest (therapy).

As used herein, the term “shuttle plasmid” is a plasmid that is capable of replicating as a plasmid in bacteria and as a phage in mycobacteria. Shuttle plasmids capable of reproduction in both bacteria and mycobacteria are known in the art (see, e.g. O'Donnell, M A et al. U.S. Pat. No. 5,591,632; O'Donnell, M A et al., U.S. Pat. No. 5,776,465; and Bloom, B R et al., U.S. Pat. No. 6,270,776).

As used herein, the term “enhanced immunostimulatory properties” means an improved, greater or augmented immune response by the recombinant bacterium of the invention as compared to an appropriate control. An exemplary control includes comparison of immunostimulatory properties of a bacterium in which a vector is not expressed, a wild-type bacterium or a wild type BCG mycobacterium.

The term “phIL2MUC1” and “pIL2MUC1 ” refer to the plasmid constructed from the pMOD12 shuttle plasmid comprising the cDNA for IL2 and the cDNA for the truncated form of MUC1 containing twenty two tandem repeats. This plasmid was grown in E. coli. Alternatively, the term “BCG-phIL2MUC1” and “BCG-pIL2MUC1” refer to the plasmid constructed from the pMOD12 shuttle plasmid comprising the cDNA for IL2 and the cDNA for the truncated form of MUC1 containing twenty two tandem repeats. This plasmid was grown in BCG.

As used herein, the term “recombinant bacterium” refers to any bacterium that has been modified by the introduction of heterologous DNA. “Wild-type” or “control” bacterium include bacterium that are substantially identical to the recombinant bacterium, but do not express one or more of the proteins encoded by the heterologous DNA, e.g. do not include or express a reporter gene construct, an antibiotic resistance gene, a cytokine, a tumor antigen, or other DNA of interest. The term is intended to include progeny of the bacterium originally modified by the introduction of heterologous DNA. In a preferred embodiment, the bacterium is a gram negative bacterial cell, and this term is intended to include all facultatively anaerobic Gram-negative cells of the family Enterobacteriaceace such as Escherichia, Shigella, Citrobacter, Salmonella, Klebsiella, Enterobacter, Erwinia, Kluyvera, Serratia, Cedecea, Morganella, Hafnia, Edwardsiella, Providencia, Proteus and Yersinia.

In another preferred embodiment, the bacterium is a gram positive bacterial cell of the genus Mycobacteriaceae such as M. bovis-BCG, M leprae, M marinum, M. smegmatis and M. tuberculosis. Particularly preferred recombinant bacterium are E. coli , M. bovis-BCG and M. smegmatis.

As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” or simply “expression vectors”. In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

As used herein, “heterologous DNA” or “heterologous nucleic acid” includes DNA that does not occur naturally as part of the genome in which it is present, or which is found in a location or locations in the genome that differs from that in which it occurs in nature. Heterologous DNA is not endogenous to the cell into which it is introduced, but has been obtained from another cell. Generally, although not necessarily, such DNA encodes RNA and proteins that are not normally produced by the cell in which it is expressed. Heterologous DNA may also be referred to as foreign DNA. Any DNA that one of skill in the art would recognize or consider as heterologous or foreign to the cell in which is expressed is herein encompassed by heterologous DNA. Examples of heterologous DNA include, but are not limited to, DNA that encodes test polypeptides, receptors, reporter genes, transcriptional and translational regulatory sequences, selectable or traceable marker proteins, such as a protein that confers drug resistance.

As used herein, the term “host cell” is intended to refer to a cell into which a nucleic acid of the invention, such as a recombinant expression vector of the invention, has been introduced. The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

As used herein, the term “appropriately expressed” is defined a expression of MUC-1 such that an immune response to MUC-1 is initiated and expression of interleukin-2 such that it a Th1 immune response is potentiated.

As used herein, the term “secreted” is intended to include an increase in the secretion of a polypeptide into the periplasmic space or into the extracellular milieu, e.g. a heterologous polypeptide, preferably a cytokine.

The term “signal peptide” or “signal sequence” refers to a peptide serves to direct a protein containing such a sequence from the endoplasmic reticulum of a cell to the golgi apparatus and ultimately to a lipid bilayer (e.g., for secretion). In a preferred embodiment, the signal peptide or signal sequence contains about 20 amino acids which occurs at the N-terminus of secretory and integral membrane proteins and which contains a large number of hydrophobic amino acid residues. For example, a signal sequence contains at least about 14-28 amino acid residues, preferably about 16-26 amino acid residues, more preferably about 18-24 amino acid residues, and more preferably about 20-22 amino acid residues, and has at least about 40-70%, preferably about 50-65%, and more preferably about 55-60% hydrophobic amino acid residues (e.g., Alanine, Valine, Leucine, Isoleucine, Phenylalanine, Tyrosine, Tryptophan, or Proline)., An example of a signal sequence includes, but is not limited to, the BCG alpha signal sequence.

As used herein, an “effective amount” of a recombinant bacterium refers to an amount of such bacterium which is effective, either alone or in combination with a pharmaceutically acceptable carrier, upon single- or multiple-dose administration to the subject, e.g., a patient, at inhibiting the growth or proliferation, inducing the killing, or preventing the growth of hyperproliferative cells. Such growth inhibition or killing can be reflected as a prolongation of the survival of the subject, e.g., a patient beyond that expected in the absence of such treatment, or any improvement in the prognosis of the subject relative to the absence of such treatment.

As used herein, the language “subject” is intended to include human and nonhuman animals. Preferred human animals include a human patient having a disorder characterized by the aberrant activity of a hyperproliferative cell. The term “nonhuman animals” of the invention includes all vertebrates, e.g. mammals and nonmammals, such as nonhuman primates, sheep, dog, cat, horse, cow, chickens, amphibians, reptiles, etc. Preferably, the subject is a human patient, e.g. a cancer patient, e.g. a patient with breast cancer.

As used herein, “inhibiting the growth or proliferation” of the hyperproliferative cell, e.g., neoplastic cell, e.g., benign hyperplastic cell, refers to slowing, interrupting, arresting or stopping its growth and metastasis, and does not necessarily indicate a total elimination of the neoplastic growth.

As used herein, “inducing the killing” of the hyperproliferative cell, e.g. neoplastic cell, e.g. benign hyperplastic cell, refers to the partial or complete elimination of such cells, and does not necessarily indicate a total elimination of the neoplastic growth.

The terms “induce”, “inhibit”, “potentiate”, “elevate”, “increase” “decrease” or the like, denote quantitative differences between two states, refer to at least statistically significant differences between the two states. For example, “an amount effective to inhibit growth of hyperproliferative cells” means that the rate of growth of the cells will at least statistically significantly different from the untreated cells. Such terms are applied herein to, for example rates of cell proliferation.

As used herein, the terms “hyperproliferative”, “hyperplastic”, “malignant” and “neoplastic” are used interchangeably, and refer to those cells in an abnormal state or condition characterized by rapid proliferation or neoplasia,. The terms are meant to include all types of hyperproliferative growth, hyperplastic growth, cancerous growths or oncogenic processes, metastatic tissues or malignantly transformed cells, tissues, or organs, irrespective of histopathologic type or stage of invasiveness. “Neoplastic disease” is characterized by malignant tumor growth or in disease states characterized by benign hyperproliferative and hyperplastic cells.

The common medical meaning of the term “neoplasia” refers to “new cell growth” that results as a loss of responsiveness to normal growth controls, e.g., neoplastic cell growth. A “hyperplasia” refers to cells undergoing an abnormally high rate of growth. However, as used herein, the terms neoplasia and hyperplasia can be used interchangeably, as their context will reveal, referring generally to cells experiencing abnormal cell growth rates. Neoplasias and hyperplasias include “tumors,” which may be either benign, premalignant or malignant.

The term “carcinoma” is recognized by those skilled in the art and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is recognized by those skilled in the art and refers to malignant tumors of mesenchymal derivation.

As used herein, the terms “leukemia” or “leukemic cancer” refers to all cancers or neoplasias of the hemopoietic and immune systems (blood and lymphatic system). These terms refer to a progressive, malignant disease of the blood-forming organs, marked by distorted proliferation and development of leukocytes and their precursors in the blood and bone marrow. Myelomas refer to other types of tumors of the blood, bone marrow cells. Lymphomas refer to tumors of the lymph tissue.

The term “cytokine” is meant to include any one of the group of hormone-like mediators produced by T and B lymphocytes. Representative cytokines include but are not limited to Interleukin-1 (IL-1), IL2, IL3, IL4, IL-5, IL-6, IL-7, IL-8, IL-9, IL-10, IL-11, IL-12, IL-13, IL-15, IL-18, Interferon gamma (IFN-γ), Tumor Necrosis Factor alpha (TNF-α), and Transforming Growth Factor-beta (TGF-β). An “active” fragment of a cytokine is a fragment of a cytokine that retains activity as determined using standard in vitro and in vivo assays. For example, assays for determining IL2 and . IFN-γ activity are known in the art (See e.g. Campos, M. (1989) Cell. Immun. 120:259-269 and Czarniecki, C. W. (1986) J. Interferon Res. 6:29-37.) Assays for determining the activity of other cytokines are known and can readily be conducted by those having ordinary skill in the art.

The term “immune response” includes any response associated with immunity including, but not limited to, increases or decreases in cytokine expression, production or secretion (e.g., IL-12, IL-10, TGFβ or TNFα expression, production or secretion), cytotoxicity, immune cell migration, antibody production and/or immune cellular responses. The phrase “modulating an immune response” or “modulation of an immune response” includes upregulation, potentiating, stimulating, enhancing or increasing an immune response, as defined herein. For example, an immune response can be upregulated, enhanced, stimulated or increased directly by use of a modulator of the present invention (e.g., a stimulatory modulator). Alternatively, a modulator can be used to “potentiate” an immune response, for example, by enhancing, stimulating or increasing immune responsiveness to a stimulatory modulator. The phrase “modulating an immune response” or “modulation of an immune response” also includes downregulation, inhibition or decreasing an immune response as defined herein.

Immune responses in a subject or patient can be further characterized as being either type-1 or type-2 immune responses.

A “type-1 immune response”, also referred to herein as a “type-1 response” or a “T helper type 1 (Th1) response” includes a response by CD4⁺ T cells that is characterized by the expression, production or secretion of one or more type-1 cytokines and that is associated with delayed type hypersensitivity responses. The phrase “type-1 cytokine” includes a cytokine that is preferentially or exclusively expressed, produced or secreted by a Th1 cell, that favors development of Th1 cells and/or that potentiates, enhances or otherwise mediates delayed type hypersensitivity reactions. Preferred type-1 cytokines include, but are not limited to, GM-CSF, IL-2, IFN-γ, TNF-α, IL-12, IL-15 and IL-18.

A “type-2 immune response”, also referred to herein as a “type-2 response or a “T helper type 2 (Th2) response” refers to a response by CD4⁺T cells that is characterized by the production of one or more type-2 cytokines and that is associated with humoral or antibody-mediated immunity (e.g., efficient B cell, “help” provided by Th2 cells, for example, leading to enhanced IgG1 and/or IgE production). The phrase “type-2 cytokine” includes a cytokine that is preferentially or exclusively expressed, produced or secreted by a Th2 cell, that favors development of Th2 cells and/or that potentiates, enhances or otherwise mediates antibody production by B lymphocytes. Preferred type-2 cytokines include, but are not limited to, IL-4, IL-5, IL-6, IL-10, and IL-13.

Cytokine expression, secretion or production modulates or further enhances or upregulates an immune response, for example, a type-1 of type-2 immune response. For example, it is known that cytokines play a dominant role in controlling the differentiation of T helper precursors (Th0) to either the Th1 or Th2 lineage. Type-1 cytokines, such as IFN-γ, can enhance the development of Th1 cells and inhibit the development of Th2 cells, whereas type-2 cytokines, such as IL-4 and IL-10, can enhance the development of Th2 cells and inhibit the development of Th1 cells. Thus, cytokines can reciprocally regulate the development and/or progression of either a type-1 or a type-2 response.

Cytokine expression, secretion or production can also be an indicator of an immune response, for example, an indicator of a type-1 or type-2 immune response.

For example, a “cytokine profile” can be indicative of a type-1 or type-2 immune response. The term “cytokine profile” includes expression, production or secretion of at least one cytokine associated with a particular type of immune response (e.g., a type-1 or type-2 immune response) and/or includes diminished or reduced expression, production or secretion of at least one cytokine associated with a mutually exclusive type of immune response (e.g., a type-2 or type-1 immune response, respectively). For example, a type-1 cytokine profile can include enhanced or increased expression, production or secretion of at least one of interleukin-2 (IL-2), interleukin-12 (IL-12), interferon-γ (IFN-γ) and tumor necrosis factor-β (TNF-β) and/or can include reduced or decreased expression, production or secretion of at least one of interleukin-4 (IL-4), interleukin-5 (IL-5), interleukin-6 (IL-6) and interleukin-10 (IL-10). Likewise, a type-2 cytokine profile can include expression, production or secretion of at least one of interleukin-4 (IL-4), interleukin-5 (IL-5), and interleukin-10 (IL-10) and/or can include reduced or decreased expression, production or secretion of at least one of interleukin-2 (IL-2), interleukin-12 (IL-12), interferon-γ (IFN-γ) and tumor necrosis factors-β (TNF-β).

The phrase “type-1 immunity” includes immunity characterized predominantly by type-1 immune responses (e.g., delayed type hypersensitivity, macrophage activation and or cellular cytotoxicity), by expression, production or secretion of at least one type-1 cytokine and/or expression of a type-1 immunity cytokine profile. The phrase “type-2 immunity” includes immunity characterized predominantly by type-2 immune responses (e.g., B cell help, IgG1 and/or IgE production, eosinophil activation, mast cell stimulation and/or macrophage deactivation), by expression, production or secretion of at least one type-2 cytokine and/or expression of a type-2 immunity cytokine profile.

The course of certain disease states is influenced by whether a predominant type-1 or type-2 response is mounted. For example, in experimental leishmania infections in mice, animals that are resistant to infection mount predominantly a type-1 immune response, whereas animals that are susceptible to progressive infection mount predominantly a type-2 immune response (Heinzel et al. (1989) J. Exp. Med. 169:59-72; and Locksley and Scott (1992) Immunoparasitology Today 1:A58-A61). In murine schistosomiasis, a switch from type-1 to type-2 immunity is observed coincident with the release of eggs into the tissues by female parasites and is associated with a worsening of the disease condition (Pearce et al. (1991) J. Exp. Med. 173:159-166; Grzych et al. (1991) J. Immunol. 141:1322-1327; and Kullberg et al. (1992) J. Immunol. 148:3264-3270). Many human diseases, including chronic infections (such as with human immunodeficiency virus (HIV) or tuberculosis) and certain metastatic carcinomas, also are characterized by a type-1 to type-2 switch. (see e.g., Shearer and Clerici (1992) Prog. Chem. Immunol. 54:21-43; Clerici and Shearer (1993) Immunol. Today 14:107-111; Yamamura et al. (1993) J. Clin. Invest. 91:1005-1010; Pisa et al. (1992) Proc. Natl. Acad. Sci. USA 89:7708-7712; Fauci (1988) Science 239:617-623). Furthermore, certain autoimmune diseases have been shown to be associated with a predominant type-1 response. For example, patients with rheumatoid arthritis have predominantly Th1 cells in synovial tissue (Simon et al. (1994) Proc. Natl. Acad. Sci. USA 91:8562-8566) and experimental autoimmune encephalomyelitis (EAE) can be induced by autoreactive Th1 cells (Kuchroo et al. (1993) J. Immunol. 151:4371-4381).

The phrase “potentiating or potentiation of a type-1 or type-2 immune response” includes upregulation, stimulation or enhancement of a type-1 or type-2 response, respectively (e.g., commitment of T helper precursors to either a Th1 or Th2 lineage, further differentiation of cells to either the Th1 or Th2 phenotype and/or continued function of Th1 or Th2 cells during an ongoing immune response). For a review of Th1 and Th2 subsets see, for example, Seder and Paul (1994) Ann. Rev. Immunol. 12:635-673.

The phrase “potentiating or potentiation of a type-1 immune response” also includes downregulation or inhibition of a type-2 immune response. The phrase “potentiating or potentiation of a type-2 immune response” also includes downregulation or inhibition of a type-1 immune response.

The term “immunomodulatory molecule”, used interchangeably herein with the term “immunomodulatory “agent” includes a molecule or agent which has a modulatory or regulatory activity which is normally associated with an immune response in an organism, for example, higher animals and humans. An activity (e.g., a biological or functional activity) associated with an immune response can be any activity associated with resistance of the organism to infection with microorganisms, response to infection or response to disease. The term “activity”, “biological activity” or “functional activity”, refers to an activity exerted by a molecule of the invention (e.g., a immunomodulatory molecule, for example, a protein, polypeptide, fragment, nucleic acid molecule, antibody, biosynthetic immunomodulatory molecule, or the like) as determined in vivo, or in vitro, according to standard techniques and/or methods such as those described in the Examples.

The term “immune cell” includes cells of the immune system which are capable of expressing, producing or secreting cytokines that regulate an immune response, for example a type-1 or type-2 immune response. Preferred immune cells include human immune cells. Exemplary preferred immune cells include, but are not limited to, macrophages, dendritic cells, T cells, B cells and neutrophils. Immune cells are also referred to herein as “immune effector cells”. The term “macrophage” includes all cells within the macrophage lineage, including monocytes, circulating macrophages, tissue macrophages, activated macrophages, and the like, from mammals (e.g., from humans). The term “T cell” (i.e., T lymphocyte) is intended to include all cells within the T cell lineage, including thymocytes, immature T cells, mature T cells and the like, from mammals (e.g., from humans).

II. Embodiments of the Invention

The present invention relates to recombinant bacterium engineered to secrete a cytokine and to express a tumor antigen. Preferably, the recombinant bacterium is an enterobacterium. In a preferred embodiment, the bacterium is E. coli.

In another embodiment, the bacterium is a mycobacterium. In a preferred embodiment, the mycobacterium is attenuated Mycobacterium bovis bacillus Calmette-Guerin (BCG) or Mycobacterium smegmatis. Because both mycobacteria have excellent adjuvant activity for induction of cell-mediated (Th1) immunity, stimulate long-term memory (immunity) and have a low mortality associated with their use, they are excellent candidates as recombinant vaccines. That is, they are excellent candidates for use as vehicles (vaccine vehicles) into which genetic material (nucleic acid) of interest (e.g. nucleic acid from another source) can be inserted and subsequently expressed.

BCG in particular has important advantages as a vaccine vehicle in that: (1) it is the only childhood vaccine currently given at birth, (2) in the past 40 years, it has had a very low incidence of adverse effects, when given as a vaccine against tuberculosis; and (3) it can be used repeatedly in an individual (e.g. in multple forms).

A further advantage of BCG in particular, as well as mycobacteria in general, is the large size of its genome (approximately 3 ×10⁶ bp in length). Because the genome is large, it is able to accommodate a large amount of DNA from another source (i.e., DNA of interest) and, thus, can be used to make a multivaccine vehicle (i.e., one carrying DNA of interest encoding protective antigens for more than one tumor).

The invention also provides for the recombinant bacterium to secrete a Th1 cytokine. Preferably the cytokine is selected from the group consisting of GM-CSF, IFN-γ, TNF-α, IL-2, IL-12, IL-15 and IL-18. In a preferred embodiment, the secreted cytokine is IL-2.

In another embodiment of the invention, the recombinant bacterium is engineered to express a tumor antigen. In a preferred embodiment, the tumor antigen is selected from the group consisting of MUC1, CEA, oncofetal antigens and tumor-associated antigens. Also included are tumor antigens encoded by a single DNA strand. In a preferred embodiment, the tumor antigen is MUC1.

In another aspect, the invention features a recombinant mycobacterium comprising a first nucleic acid molecule encoding a Th1 cytokine operatively linked to a first promoter and a mycobacterial secretion signal sequence and a second DNA molecule encoding a tumor antigen operatively linked to a second promoter, wherein the cytokine is expressed and secreted from the recombinant mycobacterium and the tumor antigen is expressed by the mycobacterium, such that the mycobacterium is capable of inducing an immune response to the tumor antigen in a subject.

In one embodiment, the recombinant bacterium comprises a first promoter which is a bacterial heat shock protein (hsp) gene promoter or a bacterial stress protein gene promoter. In a preferred embodiment, the first promoter is hsp60 or hsp70. These regulatory elements, and particularly the hsp70 promoter, are advantageously selected because they are among the most powerful in bacteria (Neidhardt, F C et al., eds. (1987) Washington D.C.: American Society for Microbiology, pp. 1654) and because hsp70 synthesis is induced to very high levels during phagocytosis of some bacteria by macrophage (Buchmeier et al. (1990) Science 248:730-732). The hsp promoters also normally control the expression of proteins that are dominant antigens in the immune response to mycobacterial infection (Young, R A, (1990) Ann Rev Immunol 8:401-420; Lamb et al. (1990) Molec. Biol. And Med. 7:311-321; and Kaufmann, S H E (1990) Immunol Today 11:129-136).

In another embodiment, the recombinant bacterium comprises a second promoter that is a bacterial heat shock gene promoter or a bacterial stress protein gene promoter. In a preferred embodiment, the first promoter is hsp60 or hsp70.

In another embodiment, the recombinant bacterium comprises a bacterial secretion signal sequence. In a preferred embodiment, the bacterial secretion sequence is the BCG alpha signal sequence

In another embodiment of the invention, the subject is a mammal. In a preferred embodiment, the subject is human.

Another aspect of the invention features a recombinant mycobacterium which comprises (a) a first DNA molecule encoding a Th1 cytokine, (b) a first promoter, (c) a mycobacterial secretion sequence, (d) a second DNA molecule encoding a tumor antigen and (e) a second promoter wherein the 5′ to 3′ order is the first promoter of (b), the secretion signal sequence of (c), the first DNA molecule (a), the second promoter of (e), the second DNA molecule of (d), wherein the expression of the first DNA molecule of (a) is under the control of the first promoter of (b) and the cytokine is expressed and secreted from the mycobacterium and the expression of the second DNA molecule of (d) is under the control of the second promoter of (e) and the tumor antigen is expressed by the mycobacterium such that the recombinant mycobacterium is capable of inducing an immune response to the tumor antigen in a subject.

Another aspect of the invention features a recombinant BCG having a dual promoter plasmid comprising (a) a first DNA molecule encoding interleukin-2, (b) a first promoter, (c) a mycobacterial secretion signal sequence, (d) a second DNA molecule encoding MUC1 and (e) a second promoter wherein the 5′ to 3′ order is the first promoter of (b), the secretion signal sequence of (c), the first DNA molecule of (a), the second promoter of (e) and the second DNA molecule of (d), wherein the expression of the first DNA molecule of (a) is under the control of the first promoter of (b) and the cytokine is expressed and secreted from the mycobacterium and the expression of the second DNA molecule of (d) is under the control of the second promoter of (e) and the tumor antigen is expressed by the mycobacterium thereby inducing an immune response to the tumor antigen in a mammalian host.

In another aspect, the invention features a recombinant bacterium encoding a tumor antigen and having enhanced immunostimulatory properties comprising a first DNA molecule encoding a cytokine and a second DNA molecule encoding a tumor antigen, wherein the first DNA molecule encoding the cytokine is under the control of a first promoter and the cytokine is secreted from the bacterium in a biologically active form and wherein the second DNA molecule encoding the tumor antigen is under the control of a second promoter and the tumor antigen is expressed by the recombinant bacteria.

In another aspect, the invention features a recombinant BCG encoding MUC1 and having enhanced immunostimulatory properties and having incorporated therein a plasmid comprising a first DNA molecule encoding interleukin-2 operably linked to a first mycobacterial heat shock protein gene promoter and a mycobacterial secretion signal sequence and a second DNA molecule encoding MUC1 operably linked to a second mycobacterial heat shock protein gene promoter wherein the 5′ to 3′ order of the plasmid is the first promoter, the secretion signal sequence, the first DNA molecule encoding the interleukin-2, the second promoter, the second DNA molecule encoding MUC1 wherein the interleukin-2 is expressed and secreted from the recombinant BCG in a biologically active form and the MUC1 is expressed by the recombinant BCG.

In another aspect, the invention features an E. coli-BCG shuttle plasmid which, when expressed in a mycobacterium, results in specificity for a tumor antigen and enhanced immunstimulatory properties, the shuttle plasmid comprises (a) a first DNA molecule encoding a Th1 cytokine, (b) a first promoter comprising DNA encoding a mycobacterial heat shock protein promoter and translational start site, (c) a mycobacterial secretion signal sequence, (d) a second DNA molecule encoding a tumor antigen, and (e) a second promoter comprising DNA encoding mycobacterial heat shock protein promoter and translational start site wherein the 5′ to 3′ order is the first promoter of (b), the secretion signal sequence of (c), the first DNA molecule of (a), the second promoter of (e) and the second DNA molecule of (d), wherein the expression of the first DNA molecule of (a) is under the control of the first promoter of (b) and the cytokine is expressed and secreted from the mycobacterium in a biologically active from and the expression of the second DNA molecule of (d) is under the control of the second promoter of (e) and the tumor antigen is expressed by the mycobacterium.

In one embodiment, the E. coli-BCG shuttle plasmid further comprises an epitope tag 5′ of the DNA encoding a T_{H}1 cytokine. In a preferred embodiment, the epitope tag is viral influenza hemagglutinin

In another embodiment, the E. coli-BCG shuttle plasmid further comprises an epitope tage 5′ of the DNA encoding a tumor antigen. In a preferred embodiment, the epitope tag is viral influenza hemagglutinin.

In another aspect, the invention features a method of inhibiting growth or proliferation of, or inducing killing of a tumor cell in a subject, comprising administering to the subject a recombinant bacterium of the present invention in an amount that is effective to inhibit growth or proliferation of, reduce the size of, or induce killing of the tumor in the subject.

In one embodiment, the subject is a mammal. In a preferred embodiment, the subject is a human.

III Therapeutic Methods

The present invention provides for both prophylactic and therapeutic methods of treating subjects (e.g., human subjects). In one aspect, the invention provides a method for preventing or treating a disease or a disorder in a subject prophylactically or therapeutically. Administration of an agent prophylactically (i.e. the recombinant bacterium of the present invention) can occur prior to the manifestation of symptoms of an undesired disease or disorder, such that the disease or disorder is prevented or, alternatively, delayed in its progression. The prophylactic methods of the present invention can be carried out in a similar manner to therapeutic methods described herein, although dosage and treatment regimes may differ.

Another aspect of the invention pertains to methods for treating a subject therapeutically. In one embodiment, the present invention includes methods of inhibiting growth or proliferation of, or inducing killing of hyperproliferative cells, e.g. tumor cells, in a subject. A preferred embodiment of the invention involves administering to a subject a recombinant mycobacterium that is engineered to secrete a Th1 cytokine and express a tumor antigen, in an amount effective to inhibit growth or proliferation of, reduce the size of, or induce killing of the tumor in the subject. Accordingly, the present method has therapeutic utility in biasing an immune response towards a type-1 immune response depending upon the desired therapeutic regimen. Such modulatory methods are particularly useful in diseases such as cancer.

Another aspect of the invention relates to a vaccine for immunizing a subject against a neoplastic disease, comprising an immunogenic amount of the recombinant bacterium engineered to secrete a cytokine and express a tumor, either alone or dispersed in a physiologically acceptable, nontoxic vehicle, which amount is effective to immunize a subject, preferably a human, against a neoplastic disease.

The human tuberculosis vaccine Mycobacterium bovis bacillus Calmette-Guerin (M. bovis-BCG or BCG) has features that make it a particularly attrractive live recombinant vaccine vehicle (Calmette et al. (1924) Bull Acad Natl Med (Paris) 91:787-796). BCG and other mycobacteria are highly effective adjuvants, and the immune response to mycobacteria has been studied extensively. With nearly 2 billion immunizations, BCG has a long record of safe use in man (Luelmo, F. (1982) Am Rev Respir Dis 125:70-72; Lotte et al. (1984) Adv. Tuberc Res 21:107-193). It is one of the few vaccines that can be given at birth, it engenders long-lived immune responses with only a single dose, and there is a worldwide distribution network with experience in BCG vaccination. Moreover, phase I trials using a synthetic MUC1 peptide admixed with BCG was has been performed and therapeutic regimens determined Goydos, JS et al. (1996) J. Surg. Res 63:298-304).

Accordingly, the vaccines are administered in a manner compatible with the dosage formulation, and in such amount as will be therapeutically effective and immunogenic. The quantity to be administered depends on the subject to be treated, capacity of the subject's immune system to generate a cellular immune response, and degree of protection desired. Precise amounts of active ingredient required to be administered depend on the judgment of the practitioner and are peculiar to each individual. However, suitable dosage ranges are of the order of about one microgram to about one milligram, preferably about 1 microgram and more preferably about 5 micrograms, and more preferably 100 micrograms active ingredient per kilogram bodyweight individual. Suitable regimes for initial administration and booster shots are also variable, but are typified by an initial administration followed in one or two week intervals by a subsequent injection or other administration.

IV Pharmaceutical Compositions

In another aspect, the present invention provides pharmaceutically acceptable compositions which comprise a therapeutically-effective amount of the recombinant bacterium engineered to secrete a cytokine and to express a tumor antigen on the bacterium cell surface either alone or in a pharmaceutically acceptable carrier.

In a preferred embodiment, as described in detail below, the pharmaceutical compositions of the invention may be specially formulated for administration in solid or liquid form, including those adapted for the following: (a) oral administration, for example, drenches (aqueous or non-aqueous solutions or suspensions), tablets, boluses, powders, granules, pastes, mouthwash, hydrogels; (b) parenteral administration, for example, by subcutaneous, intramuscular or intravenous injection as, for example, a sterile solution or suspension; (c) intracavity administration, e.g., intraperitoneal instillation, intravesical (i.e., urinary bladder) instillation, intrathecal administration, (d) intraorgan administration, e.g., intraprostatical administration, (e) topical application, for example, as a cream, ointment or spray applied to the skin; (f) intravaginal or intrarectal administration, for example, as a pessary, cream, foam, enema, suppository; or (g) aerosol, for example, as an aqueous aerosol, liposomal preparation or solid particles containing the agent(s).

In vitro assays and animal studies as described in the Examples D through K below, or an assay similar thereto (e.g., differing in choice of cells or animal used) can be used to determine an “effective amount” of the recombinant bacterium, or combinations thereof. The ordinarily skilled artisan would select an appropriate amount of each individual compound in the combination for use in the aforementioned assays or similar assays. Changes in cell activity or cell proliferation can be used to determine whether the selected amounts are “effective amount” for the particular combination of compounds. The regimen of administration also can affect what constitutes an effective amount. Further, several divided dosages, as well as staggered dosages, can be administered daily or sequentially, or the dose can be proportionally increased or decreased as indicated by the exigencies of the therapeutic situation.

The phrase “pharmaceutically acceptable” is employed herein to refer to those recombinant bacterium compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (a) sugars, such as lactose, glucose and sucrose; (b) starches, such as corn starch and potato starch; (c) cellulose, and its derivatives, such as sodium carboxymethyl cellulose, ethyl cellulose and cellulose acetate; (d) powdered tragacanth; (e) malt; (f) gelatin; (g) talc; (h) excipients, such as cocoa butter and suppository waxes; (i) oils, such as peanut oil, cottonseed oil, safflower oil, sesame oil, olive oil, corn oil and soybean oil; (j) glycols, such as propylene glycol; (k) polyols, such as glycerin, sorbitol, mannitol and polyethylene glycol; (l) esters, such as ethyl oleate and ethyl laurate; (m) agar; (n) buffering agents, such as magnesium hydroxide and aluminum hydroxide; (o) alginic acid; (p) pyrogen-free water; (q) isotonic saline; (r) Ringer's solution; (s) ethyl alcohol; (t) phosphate buffer solutions; and (u) other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (a) water soluble antioxidants, such as ascorbic acid, cysteine hydrochloride, sodium bisulfate, sodium metabisulfite, sodium sulfite and the like; (b) oil-soluble antioxidants, such as ascorbyl palmitate, butylated hydroxyanisole (BHA), butylated hydroxytoluene (BHT), lecithin, propyl gallate, alpha-tocopherol, and the like; and (c) metal chelating agents, such as citric acid, ethylenediamine tetraacetic acid (EDTA), sorbitol, tartaric acid, phosphoric acid, and the like.

Compositions containing the recombinant bacterium of the present invention may conveniently be presented in unit dosage form. The amount of active ingredient which can be combined with a carrier material to produce a single dosage form will vary depending upon the host being treated, the particular mode of administration. Generally, out of one hundred per cent, this dosage will range from about 1 per cent to about ninety-nine percent of active ingredient.

The term “unit dose” as it pertains to the inocula of the present invention refers to physically discrete units suitable as unitary dosages for animals, each unit containing a predetermined quantity of active material calculated to produce the desired immunogenic effect in association with the required diluent; i.e., carrier, or vehicle. The specifications for the novel unit dose of an inoculum of this invention are dictated by and are directly dependent on (a) the unique characteristics of the active material and the particular immunologic effect to be achieved, and (b) the limitations inherent in the art of compounding such active material for immunologic use in animals and human subjects, as disclosed in detail herein, these being features of the present invention.

Compositions of the invention suitable for oral administration may be in the form of capsules, cachets, pills, tablets, lozenges (using a flavored basis, usually sucrose and acacia or tragacanth), powders, granules, hydrogels, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), and/or as mouthwashes and the like, each containing a predetermined amount of the recombinant bacterium as an active ingredient. A compound may also be administered as a bolus, electuary or paste.

In solid dosage forms of the invention for oral administration (capsules, tablets, pills, dragees, powders, granules, hydrogels and the like), the active ingredient is mixed with one or more pharmaceutically-acceptable carriers, such as sodium citrate or dicalcium phosphate. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

The tablets, and other solid dosage forms of the pharmaceutical compositions of the present invention, such as dragees, capsules, pills, granules and hydrogels, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes, nanoparticles, hydrogels, and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the recombinant bacterium of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Suspensions, in addition to the active recombinant bacterium may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of the invention for rectal or vaginal administration may be presented as a suppository, which may be prepared by mixing one or more recombinant bacterium with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Compositions of the present invention which are suitable for vaginal administration also include pessaries, tampons, creams, gels, pastes, foams or spray formulations containing such carriers as are known in the art to be appropriate.

Dosage forms for the topical or transdermal administration of a recombinant bacteria include powders, sprays, ointments, pastes, creams, lotions, gels, solutions, patches and inhalants. The active recombinant bacteria may be mixed under sterile conditions with a pharmaceutically-acceptable carrier, and with any preservatives, buffers, or propellants which may be required.

The ointments, pastes, creams and gels may contain, in addition to a recombinant bacteria, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

The recombinant bacterium can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Transdermal patches have the added advantage of providing controlled delivery of a recombinant bacterium to the body. Such dosage forms can be made by dissolving or dispersing the agent in the proper medium. Absorption enhancers can also be used to increase the flux of the peptidomimetic across the skin. The rate of such flux can be controlled by either providing a rate controlling membrane or dispersing the peptidomimetic in a polymer matrix or gel.

Ophthalmic formulations, eye ointments, powders, solutions and the like, are also contemplated as being within the scope of this invention.

Pharmaceutical compositions of this invention suitable for parenteral and/or intracavity administration comprise one or more recombinant bacteria in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, solutes which render the formulation isotonic with the blood of the intended recipient or suspending or thickening agents.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

The term “administration,” is intended to include routes of introducing to a subject of the recombinant bacterium to perform their intended function. Examples of routes of administration which can be used include injection (subcutaneous, intravenous, parenterally, intraperitoneally, intrathecally, etc.), intravesically (i.e., urinary bladder), intraprostatically, oral, inhalation, rectal and transdermal. The pharmaceutical preparations are of course given by forms suitable for each administration route. For example, these preparations are administered in tablets or capsule form, by injection, inhalation, eye lotion, ointment, suppository, etc. The injection can be bolus or can be by continuous infusion. The recombinant bacterium can be administered alone, or in conjunction with either another agent as described above or with a pharmaceutically acceptable carrier, or both. The recombinant bacterium can be administered prior to the administration of the other agent, simultaneously with the agent, or after the administration of the agent.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically”, “peripheral administration” and “administered peripherally” as used herein mean the administration of the recombinant bacterium, such that it enters the patient's system and, thus, is subject to metabolism and other like processes.

Regardless of the route of administration selected, the recombinant bacterium, which may be used in a suitable hydrated form, and/or the pharmaceutical compositions of the present invention, are formulated into pharmaceutically-acceptable dosage forms by conventional methods known to those of skill in the art.

V. Examples

A. Vectors and primers

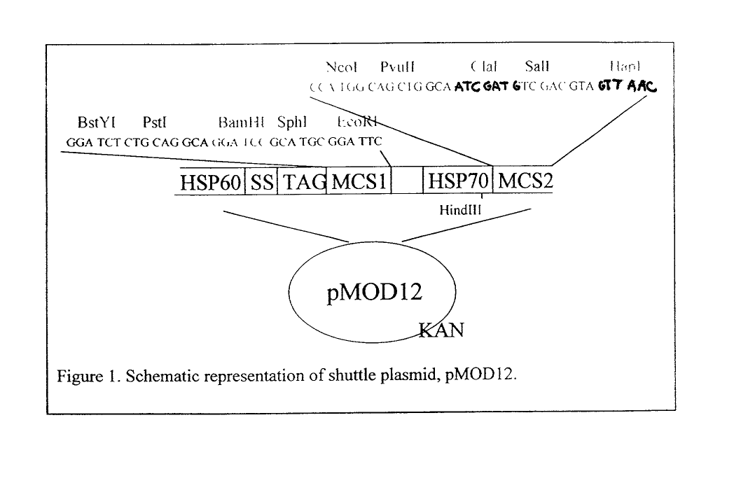

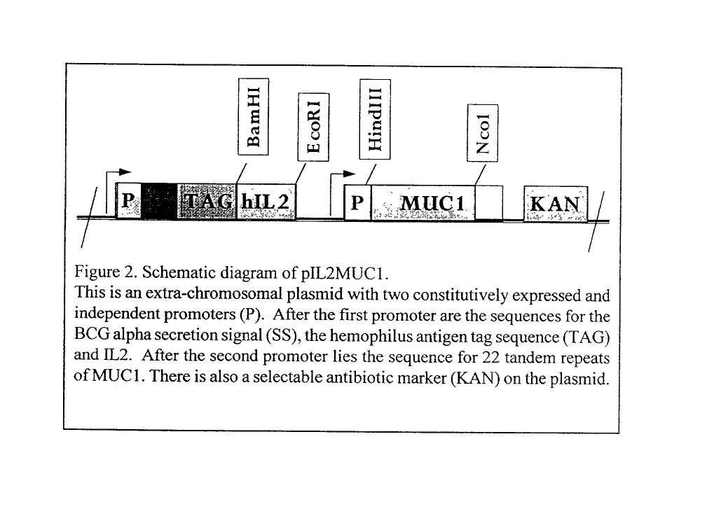

For experiments involving BCG, it is advantageous to use a shuttle plasmid capable of functioning in Escherichia coli (E. coli) and BCG. BCG replicates slowly, at 24 -36 hour intervals, and testing of the plasmid in E. coli permits more rapid experimentation. pMOD12, derived from pMV261, is a shuttle plasmid capable of replicating and expressing recombinant proteins in E. coli and BCG. pMOD12 differs from its parent plasmid in that it contains two independent and constitutively active promoters from the BCG heat shock protein 60 (HSP60) and heat shock protein 70 (HSP70), and two multiple cloning sites (MCS). A schematic diagram of pMOD12 is in FIG. 1. The first MCS comprises the sequence GGA TCT CTG CAG GCA GGA TCC GCA TGC GGA TTC (SEQ ID NO: 1) and is located after the first promoter, a start codon, the BCG alpha antigen secretion signal (SS), and a marker epitope, the influenza virus hemagglutinin (HA) epitope tag (TAG). The second MCS comprises the sequence CCA TGG CAG CTG GCA ATC GAT GTC GAC GTA GTT AAC (SEQ ID NO: 2) is located after the HSP 70 promoter. A selectable antibiotic marker for kanamycin (KAN) is also encoded by the plasmid. The plasmid, pMOD12, was genetically altered to create pIL2MUC1 , a plasmid capable of secreting IL2 and expressing a truncated form of MUC1 protein. A schematic diagram of pIL2MUC1 is illustrated in FIG. 2. The plasmid, pIL2MUC1, was constructed in a stepwise manner by inserting the cDNA for IL2 to create p12IL2 (intermediate plasmid), followed by the cDNA for the truncated form of MUC1 containing twenty two tandem repeats to create pIL2MUC1.

Several primers were used for the experiments described here. The sequences of the oligonucleotide primers used for amplification of the hIL2 DNA fragment were 5′-CAAGGGATCCGCACCTACTTCAAGTTCTACAAAG-3′ (IL2 sense) (SEQ ID NO: 3) and 5′- GCCGGAATTCTTATCAAGTTAGTGTTGAGATGAT-3′ (IL2 anti-sense) (SEQ ID NO: 4). The sequence of the primer used for sequencing pIL2MUC1 was 5′-ATTTGACAGCACACCGCCGT-3′ (SEQ ID NO: 5). All oligonucleotides were obtained from Integrated DNA Technologies, Inc, Coralville, Ia.

B. Construction of p12IL2 plasmid

The cDNA for IL2 was obtained from American Type Cell Collection (ATCC, Rockville, Md.) and amplified by the polymerase chain reaction (PCR) utilizing IL2 sense and IL2 antisense primers that contained the restriction enzyme sequences for BamHI and EcoRI, respectively. The DNA template (plasmid pcD-HT-5, ATCC, Rockville, Md.) containing the sequences for hIL2, was amplified using Taq plus long (Stratagene, La Jolla, Calif.). The temperatures used for the PCR reaction was 95° C. (denaturing for 1 minute), 55° C. (reannealing) for 1 minute and 72° C. (DNA extension) for 35 seconds for 35 cycles. The amplified DNA and pMOD12 were digested using BamHI and EcoRI, and the digested fragments ligated overnight at 16° C. using T4 ligase (New England Biolabs Inc., Beverly, Mass.). The intermediate plasmid, p12IL2, was transfected into competent bacteria (DHFα5′) using the calcium chloride method and grown on kanamycin (30 μg/ml) selectable media. Presence of the correct plasmid was documented by restriction enzyme mapping using BamHI and EcoRI.

C. Construction of pIL2MUC1 plasmid

Using p12IL2 as an intermediate vector, pIL2MUC1 was constructed by inserting a cDNA containing a truncated form of MUC1 with 22 tandem repeats through standard subcloning techniques. Briefly, a 1.7 kB fragment containing 22 tandem repeats of MUC1 was obtained from pDKOF (gift from O. J. Finn). The MUC1 1.7 kB fragment was subcloned into pl2IL2 using cohesive and blunt-end ligations. The resulting plasmid was isolated for transfection of E. coli and characterized by restriction enzyme mapping, DNA PCR amplification and DNA sequencing.

Restriction enzyme mapping was performed using BamHI, EcoRI, Hind III, and NcoI on phIL2MUC1 and pl2IL2MUC1 plasmids. Digestion of pl2IL2MUC1 or phIL12MUC1 with EcoRI resulted in linearization of the plasmids. Co-digestion of the plasmids with EcoRI and BamHI resulted in the release of a 0.5 kB fragment respresenting the DNA insert for hIL2. Digestion of phIL2MUC1 with HindIII and NcoI released a 1.7 kB fragment containing the truncated form of MUC1 with 22 tandem repeats. Co-digestion of phIL2MUC1 with all four enzymes released both DNA fragments: a 0.5 kB fragment for hIL2 and 1.7 kB representing the truncated form of MUC1 protein. A similar co-digestion of p12IL2 with all four enzymes released only the 0.5 kB fragment representing IL2.

DNA PCR amplification of BCG-hIL2MUC1 was performed using DNA extracted from BCG clones transfected with BCG-hIL2MUC1. The IL-2 DNA sequence was amplified using primers as described above in Section A: Vectors and Primers. An amplified DNA fragment measuring 0.5 kB was detected in BCG-hIL2MUC1, but not in BCG-261 or negative control.

DNA sequencing was performed by utilizing the sequencing primer for IL2 as described above in Section A: Vectors and Primers, the DNA was sequenced using the dideoxy sequencing method with fluorescent labeled dideoxynucleoside triphophosphates (Brown Sequencing Facility). After DNA verification of phIL2MUC1, BCG-hIL2MUC1 was created by transfection of this plasmid into BCG.

D. Immunodetection of IL2

Protein extracts from equivalent optical densities of cultured BCG were prepared in the following manner for one-dimensional protein electrophoresis. One ml of cultured media of BCG-hIL2MUC1 was centrifuged and the pellet resuspended in sodium dodecyl sulfate (SDS) sample and boiled for 15 minutes. The supernatant was loaded on a 15% bis-acrylamide resolving gel and the protein separated by electrophoresis. Molecular mass determinations were made by calibration of the gels with protein standards. At the completion of electrophoresis, the proteins were transferred to a 0.45 mm pore size nitrocellulose membrane (Bio-Rad Laboratories, Hercules, Calif.) and nonspecific sites blocked with 10% non-fat powdered milk in phosphate buffered saline (PBS). The presence of hIL2 was determined by immunoblotting with an anti-hIL2 antibody (Amersham Pharmacia Biotech, Piscataway, N.J.). After completion of the primary incubation, the membranes were incubated with goat anti-mouse peroxidase labeled secondary antibody. The immunoblot was developed by the enhanced chemiluminescence method (Amersham Pharmacia Biotech, Piscataway, N.J.) as directed by the manufacturer. A unique band was detected corresponding to the recombinant IL2 expressed by BCG-hIL2MUC1 as compared to the negative control (BCG-261).

To confirm that the IL2 detected arose from BCG-hIL2MUC1, a parallel Western blot was performed with a primary antibody against HA, the epitope present on the recombinant IL2 (Amersham Pharmacia Biotech, Piscataway, N.J.). Cultured media of BCG-261 was used as a negative control for these experiments. Commercially available IL2 (Boehringer Mannheim, Indianopolis, Ind.) was used as a positive control.

E. Detection of MUC1

Protein extracts from equivalent optical densities of cultured BCG were prepared in the following manner. One ml of cultured media of BCG-hIL2MUC1 was centrifuged and the pellet resuspended in SDS sample and boiled for 15 minutes. The proteins were separated on a 10% bis-acrylamide gel and transferred to nitrocellulose as described above. After blocking in a 10% milk-PBS-Tween 20 solution, the blot was incubated with an anti MUC1 antibody (anti-Episialin antibody, Sigma Immunochemicals, St. Louis, Mo.). Immunoblotting was completed in a similar manner as to that described for hIL2.

Immunoblotting with anti-MUC1 antibody of proteins extracted from BCG-hIL2-MUC1 demonstrated a unique protein of approximately 70 kD corresponding to the truncated form of MUC1 protein. Controls for this experiment included protein extracted from E. coli-phIL2MUC1 and BCG-261 (BCG containing the original plasmid, pMV261) which served as positive and negative controls, respectfully.

F. Reconstitution of SCID mice (hu-PBL-SCID mice)

The in vivo immune response to the recombinant vaccine, BCG-hIL2MUC1 to inhibit human breast cancer growth was evaluated in severe combined immunodeficient (SCID) mice reconstituted with human peripheral blood lymphocytes (hu-PBL-SCID mice). Female SCID mice (CB17 scid/scid, Taconic Farms, Inc., Germantown, N.Y.), 3-4 weeks old, were reconstituted with 50×10⁶ human peripheral blood lymphocytes (PBL) to create a xenograft of human lymphocytes in SCID mice (hu-PBL-SCID mice). PBL were procured from human buffy coats obtained from the Rhode Island Blood Center. The buffy coats were resuspended in Hanks Balanced Salt Solution (HBSS; 5.4 m Kcl, 0.3 mM Na₂HPO4, 0.4 mM KH₂PO₄, 4.2 mM NaHCO₃, 0.6 mM MgSO₄, 137 NaCl, 5.6 mM D-glucose, pH 7.4) and layered under a high density solution of Ficoll-Paque™ plus (Amersham Pharmacia Biotech, AB, Sweden) and centrifuged for 30 minutes at 1500 rpm. The interface containing the PBL was then harvested and washed twice in HBSS. The washed cell pellet was resuspended in PBS and 50×10⁶ PBL injected intraperitoneally into the SCID mice.

G. Tumor kinetics of ZR75-1 cells in hu-PBL-SCID mice

Next, the growth kinetics of ZR75-1 cancer cells in our animal model was investigated. As described earlier, SCID mice were reconstituted with 50×10⁶ human PBL on day 0 to create a xenograft of a human immune system (hu-PBL-SCID). One day after lymphocyte reconstitution of the hu-PBL-SCID mice, varying concentrations of exponentially growing ZR-75-1 cells were injected subcutaneously into the right flanks of these mice. Sham inoculation of the contralateral flank with PBS was performed. There were three groups with four mice per group. Group A received 1×10⁶ ZR75-1 cells, group B received 2×10⁶ cells and group C received 4×10⁶ cells. The mice were observed until death or 150 days, whichever was later. Outcomes of interest were time to tumor detection and size of primary tumor, and the rate and pattern of metastatic disease. At the time of necropsy, primary tissue, liver, lung and abnormal masses were harvested for histological analysis. All animals developed a gross primary tumor with the mean time to gross tumor detection inversely proportional to tumor inoculum. Fifty percent of our mice developed metastatic breast cancer irrespective of tumor inoculum. One animal (group C) developed a lymphoma and died on day 133. The results indicated that 4×10⁶ ZR75-1 cells was the suitable concentration for the tumor model described in this study.

H. Vaccination of hu-PBL-SCID mice

These experiments were designed to determine the ability of the recombinant BCG-hIL2MUC1 vaccine to prevent the development of breast cancer in the hu-PBL-SCID animal model. The vaccines used in this experiment included BCG-hIL2MUC1 (0.5 colony forming units (cfu), experimental vaccine), MUC1 peptide (100 μg, MUC1 control), BCG-261 (0.5 cfu, BCG control vaccine) and PBS (sham vaccine) diluted in a total volume of 200 μl of PBS. BCG-hIL2MUC1 is the recombinant vaccine consisting of BCG that express a truncated form of MUC1 protein with 22 tandem repeats while simultaneously secreting human IL2. MUC1 synthetic peptide was a custom peptide consisting of 5 tandem repeats of MUC1:

(GVTSAPDTRPAPGSTAPPAHGVTSAPDTRPAPGSTAPPAHGVTSAPDTR-PAPGSTAPPAHGVTSAPDTRPAPGSTAPPAHGVTSAPDTRPAPGSTAPPAH (SEQ ID NO: 6), Boston Biomolecules, Warhem, MA). BCG-261 vaccine consisted of the starting plasmid used to construct pIL2MUC1 and served as the control for BCG stimulated anti-neoplastic activity. PBS was a sham vaccine and served as an internal control. Hu-PBL-SCID mice were reconstituted as described above with 50×10⁶ PBL on day 0. One day after lymphocyte reconstitution, hu-PBL-SCID mice received 3 intra-peritoneal vaccine injections at biweekly intervals. Each group of animals had 8 mice. Two weeks after the third vaccination, 4×10⁶ ZR75-breast cancer cells were injected subcutaneously into the right flank. The mice were observed until impending death or 150 days after tumor inoculation and then sacrificed.

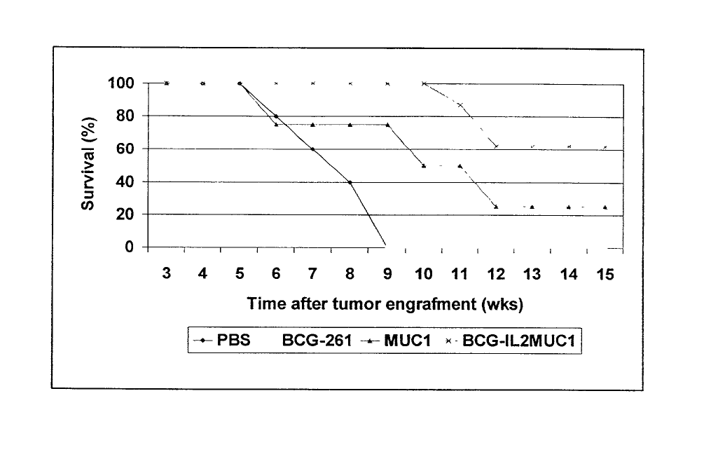

FIG. 3 shows a survival curve for tumor engrafted mice immunized with PBS, BCG-261, MUC1 or BCG-IL2MUC1. Mice immunized with BCG-IL2MUC1 had a greater rate of survival (˜60%) over a 15 week period than did those immunized with PBS (0%), BCG-261 (0%) or MUC1 (23%).

Also of interest were time to tumor detection, size of primary tumor and rate of tumor growth. Median time to tumor onset (+/−95 - percent confidence intervals) were determined for each experimental group. Mean tumor size was plotted over time after tumor engraftment for each group and polynomial regression analysis performed to determine if the rate of tumor growth differed between the groups of mice.

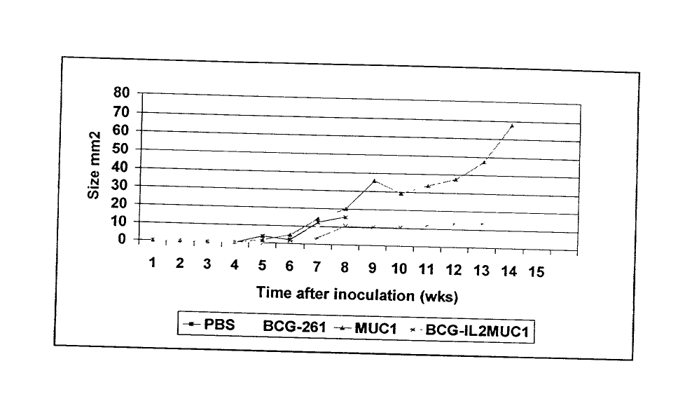

FIG. 4 depicts the rate of tumor growth in mice immunized with PBS, BCG-261, MUC1 or BCG-IL2MUC1. The size of primary tumors in control mice (PBS, MUC1 synthetic peptide and BCG-261) showed a continual increase in size over time. However, the rate of growth in the animals immunized with BCG-hIL2MUC1 was much slower than that observed in the control group. In these animals, the mean tumor size was significantly smaller (p<0.00005).

Gross examination of mice vaccinated with MUC1 or BCG-hIL2MUC1 and xenografted with human breast cancer clearly showed that seven weeks after tumor engraftment, a tumor was easily appreciated in the MUC1 immunized animal and measured >1.0 cm. The tumor on the BCG-hIL2MUC1 immunized animal was barely visible.

When the animals became gravely ill, as manifested by inability to move or groom, they were sacrificed. All surviving mice were sacrificed 24 weeks (168 days) after tumor engraftment. Three mice receiving BCG-hIL2MUC1 and one mouse receiving MUC1 peptide were sacrificed at 10 weeks after receiving the tumor xenograft to allow for completion of the experiments in a timely fashion. The mice were anesthetized by 100% CO₂ insufflation and necropsy was performed on all mice. All tumor deposits were carefully measured and fixed in a 10% formalin solution. All enlarged masses or abnormalities within the abdominal cavity or detected on the liver and spleen were harvested, measured and fixed in formalin. Liver, spleen and lung samples were also obtained. The harvested tissue was fixed in a 10% formalin solution and embedded in parrafin wax. The tissue blocks were then sectioned for hematoxylin and eosin staining and for immunohistochemistry.

I. Detection of Antibody Responses by rBCG-IL2MUC1 Immunization

Serum collected from the immunized hu-PBL-SCID mice was tested for the production of anti-MUC1 human antibodies and titer levels of anti-MUC1 human 1IgG and IgM antibodies determined by enzyme linked immunosorbant assay (ELISA) (Table I). Briefly, 96-well Immulon-4 plates were coated with 2.5 μg of MUC1 synthetic peptide, 100 mer, overnight at 40° C. After blocking with 1% bovie serum albumin (BSA), serum obtained from our immunized mice was serially diluted. Secondary antibodies used were goat anti-human Ig (for total Ig detection), goat anti-human IgM (IgM titer) and goat anti-human IgG (IgG titer) linked to horseradish peroxidase. Presence of the antibody complex was determined by incubation with 2,2′ azino-bis(3-ethylbenzo-thiazoline-6-sulfonic acid (ABTS, Boehringer Mannhein Biochemicals, Indianapolis, Ind.) and quantified at an optical density of 405 nm. These experiments were performed in duplicate.

TABLE I
Concentration of human MUC1 antibodies in mice receiving MUC1
peptide, BCG-261, BCG-hIL2MUC1, or sham vaccines. MUC1 antibodies
were detected in all groups of mice with the magnitude of
antibody lowest in the BCG-hIL2MUC1 immunized mice. The majority
of the antibodies detected in the MUC1 peptide and sham inoculated
animals were of IgM isotype.

| | | | | BCG- |
|---|---|---|---|---|
| Dilution | PBS | MUC1 | BCG-261 | hIL2MUC1 |
| 1:1 | 0.358 | 0.240 | 0.444 | 0.037 |
| 1:2 | 0.392 | 0.143 | 0.325 | 0.021 |
| 1:4 | 0.332 | 0.098 | 0.241 | 0.014 |
| 1:8 | 0.175 | 0.056 | 0.055 | 0.009 |
| 1:16 | 0.057 | 0.052 | 0.028 | 0.009 |
| 1:32 | 0.029 | 0.020 | 0.013 | |
| 1:64 | 0.039 | 0.031 | 0.010 | |
| 1:128 | 0.052 | 0.002 | 0.007 | |
| 1:256 | 0.003 | | | |

*Concentration of human MUC1 antibodies in the serum
of immunized mice

| Dilution | PBS | | MUC1 | | BCG-261 | | BCG-IL | 2MUC1 |
|---|---|---|---|---|---|---|---|---|
| 1:2 | 1.55 | 0.90 | 0.24 | 0.09 | — | 0.33 | 0.03 | 0.03 |
| 1:4 | 1.55 | 0.82 | 0.22 | 0.07 | — | 0.24 | 0.02 | 0.04 |
| 1:8 | 1.23 | 0.63 | 0.12 | 0.04 | — | 0.11 | — | 0.02 |
| 1:16 | 0.88 | 0.42 | 0.04 | 0.03 | — | 0.14 | — | — |
| 1:32 | 0.57 | 0.27 | 0.01 | 0.03 | — | 0.02 | — | — |
| 1:64 | 0.40 | 0.17 | — | 0.02 | — | 0.02 | — | — |

*Isotyping of MUC1 antibodies in the serum of immunized mice

To document that the antibodies produced by the immunized mice recognized human MUC1, serum obtained from the immunized mice was used for immunohistochemistry. Breast carcinoma, from an anonymous donor, was used for staining (gift from M. Ruhul Quddus, M.D.). Briefly, slides containing the tissue sections was deparaffinized and rehydrated with alcohol and xylene. Endogenous peroxidase activity was suppressed with a 3% hydrogen peroxide solution and non-specific antigen binding blocked by incubation with swine serum. The tissue was then incubated with serum obtained from our immunized mice. After incubation with the primary antibody, a secondary antibody consisting of a biotinylated goat anti-human IgG antibody (Chemicon International, Temecula, Calif.) was adde followed by strepavidin peroxidase. Color development followed incubation with a substrate chromogen solution (3′3′-diaminobenzidine chromogen solution). Slides were then counter-stained with hematoxylin (Meyers hematoxylin, Lillie's modification, DAKO Corp., Carpenteria, Calif.). Slides were finally dehydrated with alcohol and zylene and mounted for histological evaluation. Primary antibody staining with episialin antibody was out positive control; absence of a primary antibody was used as the negative control.

Tissue sections of a breast carcinoma were evaluated by immunostaining with serum derived from hu-PBL-SCID mice immunized with BCG-hIL2MUC1. The MUC1 protein was easily visualized on the cell surface of the cancer cells whereas areas representing pools of colloid that is associated with the colloid variant of breast cancer remained unstained.

J. Flow Cytometry of Harvested Lymphocytes

Intra-peritoneal and splenic lymphocytes were evaluated by flow cytometry to determine the cellular compartments present in the experimental and control vaccinated groups. Equal concentrations of intra-peritoneal and splenic lymphocytes were prepared in RPMI 1640. The lymphocytes were incubated with the appropriate primary antibody that was conjugated to a fluorescent marker. Detection of the antibody complexes was performed and calibrated on a Becton Dickenson (Franklin Lakes, N.J.) with the exception of the anti-CD25 antibody obtained from Immunotech, In.c (Westbrook, Me.). The antibodies used for these experiments were anti-CD45 (human leukocyte common antigen), anti-CD3, anti-CD4 anti-CD8, anti-CD19, anti-CD56 and anti-CD25. Nonspecific staining with anti-mouse IgG2a and IgG1 was used for gating the events.

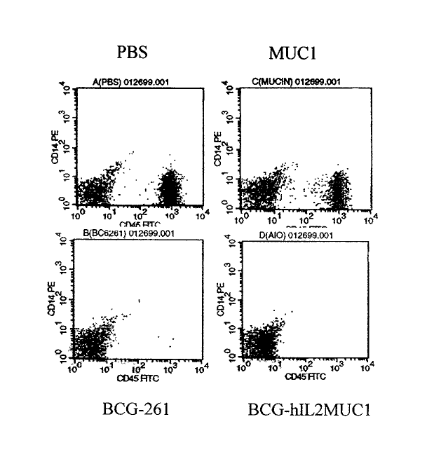

FIG. 5 shows the results of flow cytometry of peritoneal lymphocytes to detect CD45+ human lymphocytes. Flow cytometry with anti-CD45 was performed on peritoneal lymphocytes obtained from mice vaccinated with PBS (upper left), MUC1 peptide (upper right), BCG-261 (lower left) and BCG-hIL2MUC1 (lower right). CD45 positive cells were identified in the MUC1 and sham inoculated (PBS) groups of mice indicating the presence of human peritoneal lymphocytes in these animals.

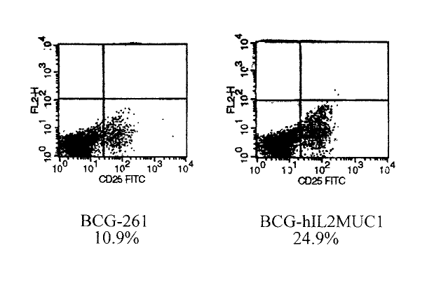

FIG. 6 shows the results of splenic lymphocytes obtained from animals vaccinated with BCG-261 or BCG-hIL2MUC1, stained with anti-CD25 and analyzed by flow cytometry. There was increased expression of CD25 positive cells in animals vaccinated with rBCG-hIL2MUC1 relative to that observed in animals vaccinated with BCG-261. The increased proportion of CD25 positive cells may be indicative of T cell activation and is associated with a Th1 immune response.

Lymphocytes obtained from peritoneal lavage and the spleen were quantified (Table II). Six weeks after reconstitution of SCID mice with human PBL by peritoneal injection, it was possible to detect human lymphocytes by peritoneal lavage. Only 2-4% of the PBL originally injected into the peritoneal cavity could be harvested in the PBS-MUC1 peptide vaccinated animals. Less than 1% of PBL could be garnered by peritoneal lavage in the animals vaccinated with BCG-261 or BCG-hIL2MUC1. Evaluation of lymphocytes from the spleen showed that there had been migration of the intra peritoneal lymphocytes into this organ. However, in addition to PBL migration to the spleen, there was marked proliferation of lymphocytes in this organ in the animals vaccinated with BCG-261 and BCG-hIL2MUC1.

TABLE II
Proliferation and shift of lymphocytes in response to vaccination
with BCG-hIL2MUC1.

| Vaccine | Peritoneum | Spleen | Total |
|---|---|---|---|
| PBS | 2.26 × 10⁶ | 10.6 × 10⁶ | 12.9 × 10⁶ |
| MUC1 Peptide | 1.31 × 10⁶ | 3.0 × 10⁶ | 4.3 × 10⁶ |
| BCG-261 | 0.34 × 10⁶ | 32.0 × 10⁶ | 32.3 × 10⁶ |
| BCG-hIL2MUC1 | 0.11 × 10⁶ | 35.4 × 10⁶ | 35.5 × 10⁶ |

K. Histological Analysis of Tissue Samples

Immunohistochemistry of tissue sections was performed using LSAB+ peroxidase kit (DAKO Corporation, Carpenteria, CA). Briefly, slides containing the tissue sections was deparafinized and rehydrated with alcohol and xylene. Endogenous peroxidase activity was suppressed with a 3% hydrogen peroxide solution and non-specific antigen binding blocked by incubation with swine serum. The tissue was then incubated with the appropriate primary antibodies. The following antibodies were used: for detection of MUC1, anti-episialin antibody (Sigma Immunochemicals, St. Louis, Mo.); for detection of cytokeratins, anti-cytokeratin, clone AE1/AE2 antibody (DAKO Corporation, Carpenteria, Calif.) and anti-CD45 antibody for human lymphocytes (DAKO Corporation, Carpenteria, Calif.). Incubation of tissue sections with murine derived control IgG served as the negative control for these experiments ((DAKO Corporation, Carpenteria, Calif.). After incubation with the primary antibody, the secondary antibody consisting of a biotinylated anti rabbit, mouse and goat immunoglobin antibody was used. Streptavidin peroxidase was then added. Color development followed incubation with a substrate chomogen solution (3′3′-diaminobenzidine chromogen solution). Slides were then counter-stained with hematoxylin (Mayers hematoxylin , Lillie's modification, DAKO Corporation, Carpenteria, Calif.). Slides were finally dehydated with alcohol and xylene and mounted for histological evaluation. Results are described in FIGS. 13-16.

Histology of primary tumors in BCG-hIL2MUC1 vaccinated animals were analyzed. In BCG-hIL2MUC1 vaccinated animals, a large area of single cell necrosis in tumors was obsrved, suggesting that part of the tumor was undergoing apoptosis. Pyknotic nuclei indicative of single cell necrosis and tumor infiltrating lymphocytes were also observed.

Expression of MUC1 in human breast cancers grown in hu-PBL-SCID mice was examined by staining primary breast cancers for MUC1 . Primary tumors xenografted in MUC1 peptide, BCG-261 and sham vaccinated mice were strongly positive for MUC1. Primary tumors xenografted in BCG-hIL2MUC1 vaccinated mice were weakly positive for MUC1 protein suggesting that the vaccine had resulted in the elimination of MUC1 positive cells.

Immunohistochemistry was also performed on the intra-abdominal masses of hu-PBL-SCID mice. Large intra-abdominal and retroperitoneal masses were detected in the sham and MUC1 peptide vaccinated animals having a histological pattern consistent with a lymphoma. The masses were also strongly positive for CD45, human common leukocyte antigen, confirming that they originated from human lymphocytes.

Immunohistochemistry analysis on hu-PBL-SCID mice vaccinated with BCG-hIL2MUC1 showed that BCG-hIL2MUC1 retained its plasmid without antibiotic selection. Administration of BCG-hIL2MUC1 resulted in the development of granulomas on the spleen, lung and liver that were strongly positive for mycobacteria.

Incorporation By Reference

The entire contents of all patents, published patent applications and other references cited herein are hereby expressly incorporated herein in their entireties by reference.

Equivalents

Those skilled in the art will recognize, or be able to ascertain using no more than routine experimentation, numerous equivalents to the specific procedures described herein. Such equivalents were considered to be within the scope of this invention and are covered by the following claims. The contents of all references, issued patents, and published patent applications cited throughout this application are hereby incorporated by reference.

We claim:

1. A recombinant bacterium engineered to secrete a cytokine and to express a tumor antigen.

2. The recombinant bacterium of claim 1, wherein said bacterium is E. coli.

3. The recombinant bacterium of claim 1, wherein said bacterium is a mycobacterium.

4. The recombinant bacterium of claim 3, wherein said bacterium is attenuated Mycobacterium bovis, bacillus Calmette Guerin (BCG).

5. The recombinant bacterium of claim 1, wherein said cytokine is a Th1 cytokine.

6. The recombinant bacterium of claim 5, wherein said Th1 cytokine is selected from the group consisting of GM-CSF, IFN-γ, TNF-α, IL-2, IL-12, IL-15 and IL-18

7. The recombinant bacterium of claim 6, wherein said Th1 cytokine is IL-2.

8. The recombinant bacterium of claim 1, wherein said tumor antigen is selected from the group consisting of MUC1, CEA, oncofetal antigens and tumor-associated antigens

9. The recombinant bacterium of claim 8, wherein said tumor antigen is MUC1.

10. A recombinant mycobacterium comprising a first nucleic acid molecule encoding a Th1 cytokine operatively linked to a first promoter and a mycobacterial secretion signal sequence and a second DNA molecule encoding a tumor antigen operatively linked to a second promoter, wherein said cytokine is expressed and secreted from said mycobacterium and said tumor antigen is expressed by said mycobacterium, such that said recombinant mycobacterium is capable of inducing an immune response to said tumor antigen in a subject.

11. The recombinant bacterium of claim 10, wherein said first promoter is a bacterial heat shock protein (hsp) gene promoter or a bacterial stress protein gene promoter.

12. The recombinant bacterium of claim 11, wherein said first promoter is hsp60 or hsp70.

13. The recombinant bacterium of claim 10, wherein said second promoter is a bacterial heat shock gene promoter or a bacterial stress protein gene promoter.

14. The recombinant bacterium of claim 13, wherein said second promoter is hsp60or hsp70.

15. The recombinant bacterium of claim 10, wherein said secretion signal sequence is the BCG alpha antigen signal sequence.

16. A recombinant mycobacterium comprising:
(a) a first DNA molecule encoding a Th1 cytokine;
(b) a first promoter;
(c) a mycobacterial secretion sequence;
(d) a second DNA molecule encoding a tumor antigen; and
(e) a second promoter;
wherein the 5′ to 3′ order is said first promoter of (b), said secretion signal sequence of (c), said first DNA molecule of (a), said second promoter of (e) and said second DNA molecule of (d), wherein the expression of said first DNA molecule of (a) is under the control of said first promoter of (b) and said cytokine is expressed and secreted from said mycobacterium and the expression of said second DNA molecule of (d) is under the control of said second promoter of (e) and said tumor antigen is expressed by said mycobacterium such that said recombinant mycobacterium is capable of inducing an immune response to said tumor antigen in a subject.

17. A recombinant BCG having a dual promoter plasmid comprising:
(a) a first DNA molecule encoding interleukin-2;
(b) a first promoter;
(c) a mycobacterial secretion signal sequence;
(d) a second DNA molecule encoding MUC1; and
(e) a second promoter;
wherein the 5′ to 3′ order is said first promoter of (b), said secretion signal sequence of (c), said first DNA molecule of (a), said second promoter of (e) and said second DNA molecule of (d), wherein the expression of said first DNA molecule of (a) is under the control of said first promoter of (b) and the cytokine expressed and secreted from the mycobacterium and the expression of said second DNA molecule of (d) is under the control of said second promoter of (e) and the tumor antigen is expressed by said mycobacterium thereby inducing an immune response to said tumor antigen in a mammalian host.

18. A recombinant bacterium encoding a tumor antigen and having enhanced immunostimulatory properties comprising a first DNA molecule encoding a cytokine and a second DNA molecule encoding a tumor antigen, wherein said first DNA molecule encoding said cytokine is under the control of a first promoter and said cytokine is secreted from said bacterium in a biologically active form and wherein said second DNA molecule encoding said tumor antigen is under the control of a second promoter and said tumor antigen is expressed by said recombinant bacterium.

19. A recombinant BCG encoding MUC1 and having enhanced immunostimulatory properties and having incorporated therein a plasmid comprising a first DNA molecule encoding interleukin-2 operably linked to a first mycobacterial heat shock protein gene promoter and a mycobacterial secretion signal sequence and a second DNA molecule encoding MUC1 operably linked to a second mycobacterial heat shock protein gene promoter wherein the 5′ to 3′ order of said plasmid is said first promoter, said secretion signal sequence, said first DNA molecule encoding said interleukin-2 , said second promoter, said second DNA molecule encoding MUC1 wherein said interleukin-2 is expressed and secreted from said recombinant BCG in a biologically active form and said MUC1 is expressed by said recombinant BCG.

20. An E coli-BCG shuttle plasmid which, when expressed in a mycobacterium, results in specificity for a tumor antigen and enhanced immunostimulatory properties, said shuttle plasmid comprising:
(a) a first DNA molecule encoding a Th1 cytokine;
(b) a first promoter comprising DNA encoding a mycobacterial heat shock protein promoter and translational start site;
(c) a mycobacterial secretion signal sequence;
(d) a second DNA molecule encoding a tumor antigen; and
(e) a second promoter comprising DNA encoding mycobacterial heat shock protein promoter and translational start site;
wherein the 5′ to 3′ order is said first promoter of (b), said secretion signal sequence of (c), said first DNA molecule of (a), said second promoter of (e) and said second DNA molecule of (d), wherein the expression of said first DNA molecule of (a) is under the control of said first promoter of (b) and the cytokine is expressed and secreted from said mycobacterium in a biologically active form and the expression of said second DNA molecule of (d) is under the control of said second promoter of (e) and the tumor antigen is expressed by said mycobacterium.

21. The E. coli-BCG shuttle plasmid of claim 20 further comprising an epitope tag 5′ of said DNA encoding a Th1 cytokine.

22. The E. coli-BCG shuttle plasmid of claim 21, wherein said epitope tag is viral influenza hemagglutinin.

23. The E. coli-BCG shuttle plasmid of claim 20 further comprising an epitope tag 5′ of said DNA encoding a tumor antigen.

24. The E. coli-BCG shuttle plasmid of claim 23, wherein said epitope tag is viral influenza hemagglutinin.

25. A method of inhibiting growth or proliferation of, or inducing killing of a tumor in a subject, comprising administering to said subject a recombinant bacterium of any one of claims 1, 10, 16, 17, 18 or 19 in an amount that is effective to inhibit growth or proliferation of, reduce the size of, or induce killing of the tumor in the subject.

26. The method of claim 25, wherein said subject is a mammal

27. The method of claim 25, wherein said subject is a human.

28. A method of preventing the formation of a tumor in a subject comprising administering to said subject a recombinant bacterium of any of claims 1, 10, 16, 17, 18 or 19 in an amount that is effective to prevent the formation of said tumor in said subject.

29. A method for stimulating an immune response to an immunogenic protein or fragment thereof, in a subject, comprising administering an effective amount of a recombinant bacterium of any one of claims 1, 10, 16, 17, 18 or 19 such that an immune response is stimulated against said immunogenic protein in said subject.

30. A method of treating a subject with cancer comprising administering to said subject an effective amount of the recombinant bacterium of any one of claims 1, 10, 16, 17, 18 or 19 such that said cancer is treated.

31. The method of claim 29, wherein said cancer is selected from the group consisting of breast cancer, prostate cancer, colon cancer, lung cancer, pancreatic cancer and ovarian cancer.

32. The method of claim 31, wherein said cancer is breast cancer.

33. A vaccine for immunizing a subject against a neoplastic disease, comprising a recombinant bacterium of any one of claims 1, 10, 16, 17, 18 or 19 and a pharmaceutically acceptable carrier therefor, wherein the recombinant bacterium in an amount effective to immunize a subject against a neoplastic disease.

34. A pharmaceutical composition comprising a recombinant bacterium of any one of claims 1, 10, 16, 17, 18 or 19 and a pharmaceutically acceptable carrier.

35. A kit for immunizing a subject against a neoplastic disease comprising the vaccine of claim 33 and instructions for use.

36. A kit for treating cancer in a subject comprising the pharmaceutical composition of claim 34 and instructions for use.


SEQUENCE LISTING (6 sequences)

SEQ ID NO: 1 - DNA, 33 bp, Escherichia coli
ggatctctgcaggcaggatccgcatgcggattc

SEQ ID NO: 2 - DNA, 36 bp, Escherichia coli
ccatggcagctggcaatcgatgtcgacgtagttaac

SEQ ID NO: 3 - DNA, 34 bp, Homo sapiens
caagggatccgcacctacttcaagttctacaaag

SEQ ID NO: 4 - DNA, 34 bp, Homo sapiens
gccggaattcttatcaagttagtgttgagatgat

SEQ ID NO: 5 - DNA, 20 bp, Escherichia coli
atttgacagcacaccgccgt

SEQ ID NO: 6 - protein, 100 aa, Homo sapiens
GVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAH